(19) United States
(12) Patent Application Publication
(10) Pub. No.: US 2022/0167698 A1
(43) Pub. Date: Jun. 2, 2022

(54) UPPER BODY OF GARMENT

(71) Applicant: Toratani Co., Ltd., Kahoku-shi, Ishikawa (JP)

(72) Inventor: Ikuo Toratani, Kahoku-shi, Ishikawa (JP)

(21) Appl. No.: 17/433,454

(22) PCT Filed: May 21, 2019
(86) PCT No.: PCT/JP2019/020059

§ 371 Date: Aug. 24, 2021

(51) Int. Cl. A41D 27/10; A41D 27/06

(52) U.S. Cl. CPC A41D 27/10; A41D 27/06

(57) ABSTRACT

A restricting portion is located between rear surfaces of left and right sleeves and prevents left and right shoulder blades of a wearer from moving apart from each other. A first seam point, which is the lowest point on a seam between a front surface of the left sleeve and a front body, is positioned above a first bottom point, which is the lowest point on a seam between the rear surface of the left sleeve and the rear body. A second seam point, which is the lowest point on a seam between a front surface of the right sleeve and the front body, is positioned above a second bottom point, which is the lowest point on a seam between the rear surface of the right sleeve and the rear body.

### TECHNICAL FIELD

The invention relates to upper bodies of garments.

### BACKGROUND ART

An upper body of a garment is known, which can cover at least a portion of the upper body of a wearer. See Patent Literature 1, for example.

### CITATION LIST

### Patent Literature

Patent Literature 1: JP 2014-196587 A

### SUMMARY OF INVENTION

When operating a mobile device such as a smartphone, a person tends to move his/her shoulder forward from a position in a good posture to put his/her arm holding the mobile device in front of his/her trunk. The term “good posture” concretely means a posture with which a person standing on a flat surface, when viewed side on, puts the root of his/her neck and his/her shoulders, elbows, and ankles in a substantially straight line. Frequent repetition or long-time maintenance of a posture in which the shoulders are placed in front of the position in the good posture can lead to deterioration of the body shape such as rounded shoulders or kyphosis. The term “rounded shoulders” means a body shape in which shoulder blades are placed far apart from each other to rotate the shoulders inward. The term “kyphosis” means a body shape in which the spine has an excessive forward curvature to put the head in a forward position. These body shapes can cause pain such as stiff shoulders.

Some types of garments have an ability to generate an enhanced stretching force between rear surfaces of both sleeves in order to make a wearer keep the good posture or correct his/her deteriorated posture. Since the enhanced stretching force prevents the wearer's shoulder blades from moving apart from each other, it is difficult for the wearer to adopt the posture in which the shoulders extend forward. This inhibits the wearer from adopting a bad posture such as rounded shoulders or kyphosis, and in addition, corrects the posture of the wearer with a deteriorated body shape. However, an excessive stretching force between rear surfaces of both sleeves can prevent the movement of not only the shoulder blades but also the arms. In particular, difficulty in rotating the arms around the shoulders and raising them forward from the sides of the trunk, i.e. difficulty in flexion of the arms can disrupt the wearer's daily life.

An object of the invention is to solve the above-mentioned problems, in particular, to provide an upper body of a garment that can make it difficult for a wearer to adopt the posture in which the shoulders extend forward to put the arms in front of the trunk, and in addition that, in order not to disrupt the wearer's daily life, can make it relatively easy for the wearer to rotate the arms around the shoulders and raise them forward from the sides of the trunk.

An upper body of a garment according to one aspect of the invention includes a body including a front body and a rear body, a left sleeve, and a right sleeve. Between the rear surfaces of the left and right sleeves, a restricting portion is located and prevents left and right shoulder blades of a wearer from moving apart from each other. The left sleeve includes a first seam point that is the lowest point on a seam between a front surface of the left sleeve and the front body, and a first bottom point that is the lowest point on a seam between a rear surface of the left sleeve and the rear body. The first bottom point is positioned below the first seam point. The right sleeve includes a second seam point that is the lowest point on a seam between a front surface of the right sleeve and the front body, and a second bottom point that is the lowest point on a seam between a rear surface of the right sleeve and the rear body. The second bottom point is positioned below the second seam point.

The restricting portion, when stretched by the wearer's shoulders, exerts tensile forces as resistance to prevent the wearer's shoulder blades from moving apart from each other. This makes it difficult for the wearer to adopt the posture in which the shoulders extend forward. On the other hand, since the first bottom point of the rear surface of the left sleeve is positioned below the first seam point of the front surface of the left sleeve, the fabric of the left sleeve can have an enlarged region extending within the rear surface of the left sleeve from the height of the first bottom point to the height of the first seam point; the enlarged region enables the wearer to rotate the left arm around the left shoulder and raise it forward from the left side of the trunk. In addition, since the second bottom point of the rear surface of the right sleeve is positioned below the second seam point of the front surface of the right sleeve, the fabric of the right sleeve can have an enlarged region extending within the rear surface of the right sleeve from the height of the second bottom point to the height of the second seam point; the enlarged region enables the wearer to rotate the right arm around the right shoulder and raise it forward from the right side of the trunk. Due to the presence of these enlarged regions, the garment can, without seriously harming the effect of the tensile force by the restricting portion, make it relatively easy for the wearer to rotate the arms around the shoulders and raise them forward from the sides of the trunk.

The restricting portion consists of a hardly-, or non-stretchy fabric, for example. The hardly-stretchy fabric, when stretched by 20% in left and right direction, increases a stretching force to 45 cN or more in the same directions. The non-stretchy fabric is stretched by less than 20% in left and right direction when experiencing a stretching force of 45 cN or more in the same directions. Note that the “stretching force” is a force that a constant-rate-loading tensile machine needs to stretch a piece of fabric, which is 10 cm long and 2.5 cm wide, in its longitudinal direction at a rate of 30 cm/min under the condition of about 20 degrees Celsius, i.e. 18-22 degrees Celsius. As the constant-rate-loading tensile machine, an AGS-X manufactured by Shimadzu Corporation was used to measure values of stretching force described herein.

The restricting portion may include a region of the rear body from the boundary between the rear body and the left sleeve to the boundary between the rear body and the right sleeve. That is, this region of the rear body may be integrated with the restricting portion. The words “integrated with” may mean that the fabric of the restricting portion is sewn and connected to the fabric of one or more of other regions of the rear body, or that the restricting portion and one or more of other regions of the rear body are woven into a single fabric. Alternatively, the restricting portion may include an additional fabric attached to the rear body and different from the fabric of the rear body. This additional fabric may be sewn on the boundary between each rear surface of the left and right sleeves and the rear body, or on each rear surface of the left and right sleeves.

The left sleeve may include a left connecting portion and a hardly-stretchy region. The left connecting portion is a region connected to a left side of the body. The hardly-stretchy region is a region that, when stretched by 20% in the longitudinal direction of the left sleeve, increases a stretching force to 45 cN or more in the same direction and that may extend at least within the rear surface of the left connecting portion. The right sleeve may include a right connecting portion and a hardly-stretchy region. The right connecting portion is a region connected to a right side of the body. The hardly-stretchy region is a region that, when stretched by 20% in the longitudinal direction of the right sleeve, increases a stretching force to 45 cN or more in the same direction and that may extend at least within the rear surface of the right connecting portion. The hardly-stretchy region of the left sleeve may range from the rear surface of the left connecting portion to a front surface thereof. The hardly-stretchy region of the right sleeve may range from the rear surface of the right connecting portion to a front surface thereof. According to these structures, each of the left and right connecting portions generates a high tensile force once it is stretched, and thus it hardly or never reduces the tensile force by the restricting portion. As a result, the garment can more effectively prevent the forward motion of the wearer's shoulder blades, upper arms, and shoulders.

Instead of the hardly-stretchy region, the left and right sleeves may each include a non-stretchy region, which is stretched by less than 20% in the longitudinal direction of the left or right sleeve when experiencing a stretching force of 45 cN or more in the same direction. The non-stretchy regions of the left and right sleeves may extend at least within the rear surfaces of the left and right connecting portions, respectively. The non-stretchy region of the left sleeve may range from the rear surface of the left connecting portion to a front surface thereof. The non-stretchy region of the right sleeve may range from the rear surface of the right connecting portion to a front surface thereof. According to these structures, any of the left and right connecting portions is hardly or not substantially stretched, thus hardly or never reducing the tensile force by the restricting portion. As a result, the garment can more effectively prevent the forward motion of the wearer's shoulder blades, upper arms, and shoulders.

In the upper body of the garment according to the above-described aspect of the invention, the restricting portion prevents the wearer's shoulder blades from moving apart from each other. On the other hand, the first bottom point is positioned below the first seam point between the left sleeve and the body, and the second bottom point is positioned below the second seam point between the right sleeve and the body. Thus, below the wearer's armpits, stretching of the fabric of the front body reduces the tensile force by the restricting portion. Accordingly, the upper body of the garment can make it difficult for the wearer to adopt the posture in which the shoulders extend forward to put the arms in front of the trunk, and in addition, enables the arms to be easily flexed at the shoulders.

### BRIEF DESCRIPTION OF DRAWINGS

FIG. 1 is a front elevation view of an upper body of a garment according to embodiment 1 of the invention;

FIG. 2 is a rear elevation view of the upper body of the garment shown in FIG. 1;

FIG. 3 is an enlarged view of an upper portion of FIG. 2;

FIG. 4 is a partial development view of the left armhole and its vicinity in the front and rear bodies;

FIG. 5 is a partial development view of the left armhole and its vicinity in the front body and the left sleeve;

FIG. 6 is a partial development view of the left armhole and its vicinity in the rear body and the left sleeve;

FIG. 7 is a plan view of a solid shape of the upper body of the garment shown in FIG. 1;

FIG. 8 is a rear elevation view of an upper body of a garment according to embodiment 2 of the invention;

FIG. 9 is a rear elevation view of a first modification of the upper body of the garment according to embodiment 2 of the invention;

FIG. 10 is a rear elevation view of a second modification of the upper body of the garment according to embodiment 2 of the invention; and

FIG. 11 is a rear elevation view of a third modification of the upper body of the garment according to embodiment 2 of the invention.

### DESCRIPTION OF EMBODIMENTS

Embodiments of the invention will be explained with reference to the drawings.

### Embodiment 1

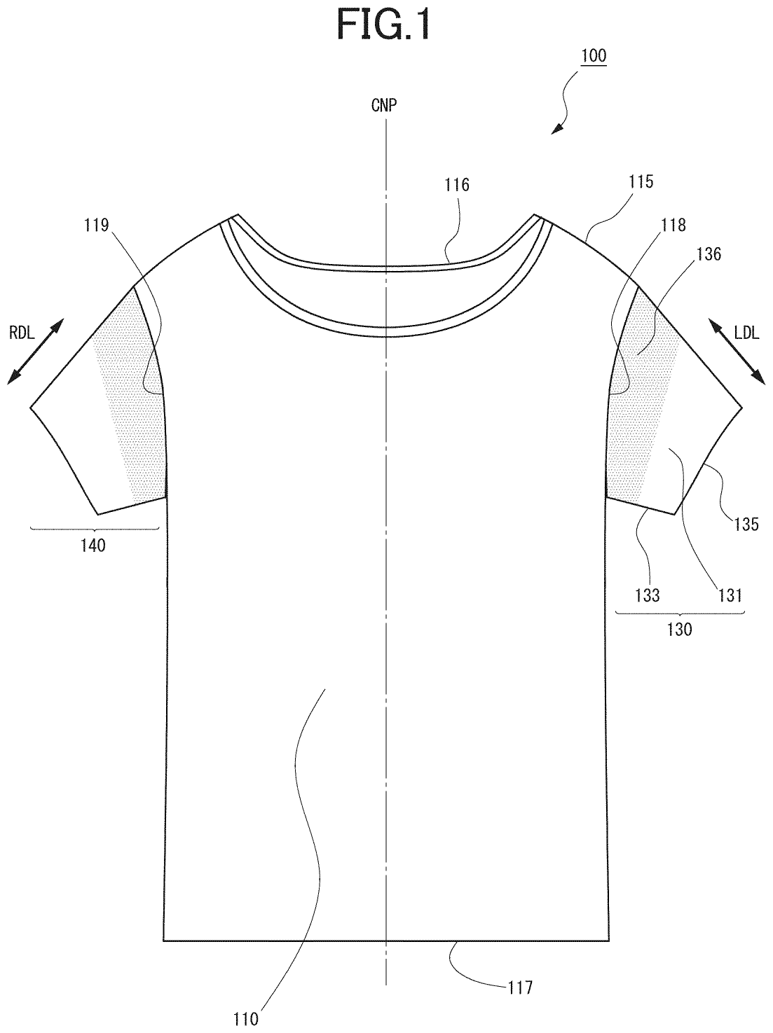

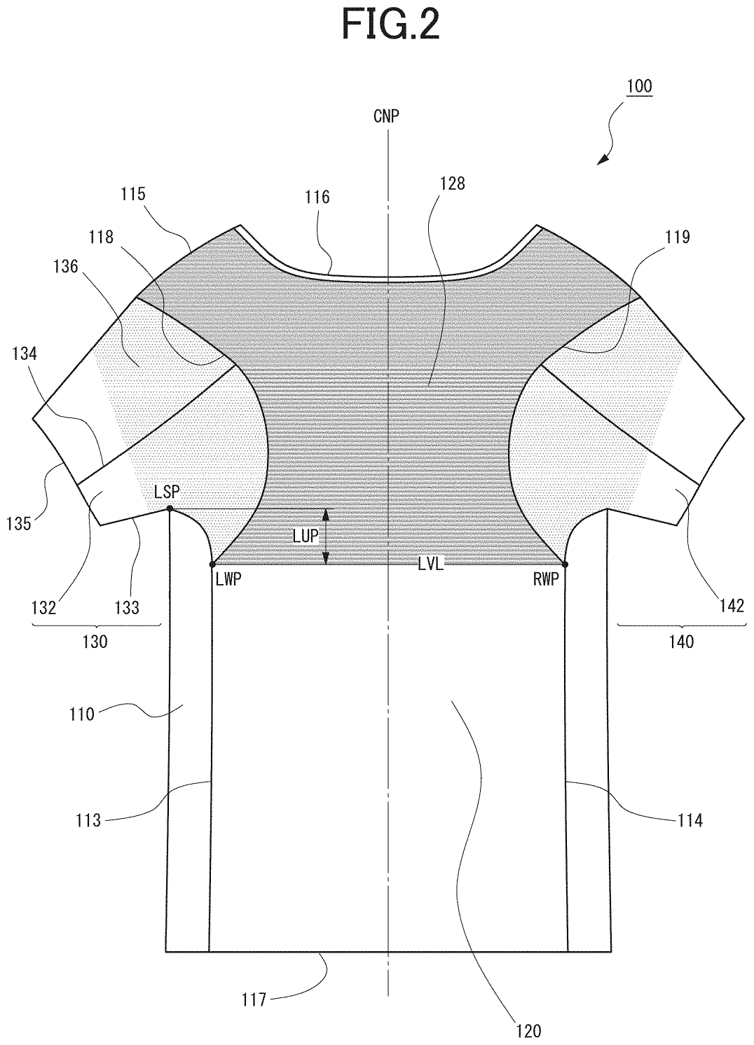

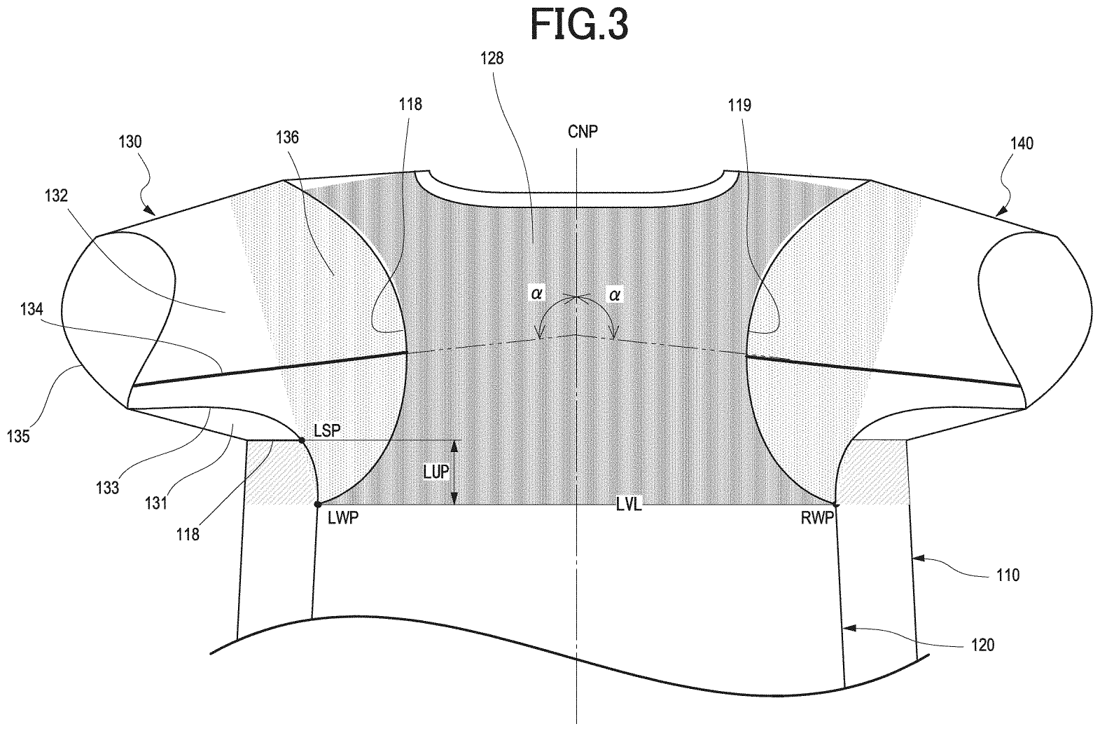

FIGS. 1 and 2 are respectively front and rear elevation views of an upper body of a garment 100 according to embodiment 1 of the invention. FIG. 3 is an enlarged view of an upper portion of FIG. 2. Hereinafter, the upper body of the garment 100 is referred to as “garment upper body,” and front-back and left-right directions are determined with respect to a wearer of the garment upper body 100. For example, “left directions” are shown as a right direction in FIG. 1, while shown as a left direction in FIG. 2.

The garment upper body 100 is configured to wrap an upper portion of the wearer's trunk and his/her upper arms. The garment upper body 100 may be used as an undergarment (innerwear) or as an intermediate or outer garment (outerwear). According to embodiment 1, the garment upper body 100 is a short-sleeved shirt.

The garment upper body 100 includes a body 110, 120, a left sleeve 130, and a right sleeve 140. The body 110, 120 has a cylindrical shape to wrap the upper portion of the wearer's trunk. The body includes a front body 110 and a rear body 120. The front body 110 is a portion of the body that faces a front surface of the upper portion of the wearer's trunk, i.e. the wearer's chest and belly and covers the almost entirety of the front surface. The rear body 120 is a portion of the body that faces a rear surface of the upper portion of the wearer's trunk, i.e. the wearer's back and covers the almost entirety of the rear surface. The front body 110 and the rear body 120 each have a bilaterally symmetrical shape. The left sleeve 130 and the right sleeve 140 are provided on left and right sides of the body, respectively. Each of the sleeves 130 and 140 has a cylindrical shape narrower than that of the body 110, 120 to wrap an upper portion of the wearer's left or right upper arm. The sleeves 130 and 140 are symmetric to a center plane CNP that bisects the body 110, 120 in left and right direction. Each size of the sleeves 130 and 140 can be freely designed in a longitudinal direction thereof (the direction of an arrow LDL or RDL shown in FIG. 1). In embodiment 1, both the sleeves 130 and 140 are designed as a short sleeve. Alternatively, the sleeves 130 and 140 may be designed as a long or three-quarter sleeve.

Instead of the two parts, i.e. the front body 110 and the rear body 120, the body of the garment upper body 100 may consist of four parts including a front body, left and right side bodies, and a rear body. Applying the invention to the four-part body only needs consideration of the side bodies as portions of the front body.

The front body 110 and the rear body 120 are sewn on each other to form a left side seam 113, a right side seam 114, and shoulder seams 115. The body thus includes a neckline 116, a hem 117, a left armhole 118, and a right armhole 119. The neckline 116 corresponds to an upper open end of the cylindrical shape of the body, and the hem 117 corresponds to a lower open end thereof. The left armhole 118 is the rim of a hole opened at an upper left side of the body, and the right armhole 119 is the rim of a hole opened at the upper right side of the body. On the left armhole 118, a proximal end of the left sleeve 130, i.e. a sleeve cap thereof is sewn, while on the right armhole 119, a sleeve cap of the right sleeve 140 is sewn.

The fabric constituting the front body 110 may be extensible or not. The fabric constituting the rear body 120 is extensible, but at least a region 128 of the rear body 120 between the left sleeve 130 and the right sleeve 140 (cf. a densely dotted region shown in FIG. 2) consists of hardly- or non-stretchy fabric. The term “hardly-stretchy” means that the fabric is hardly stretched, or more concretely, the fabric increases a stretching force to 45 cN or more in left and right direction when being stretched by 20% in the same directions. The term “non-stretchy” means that the fabric is almost or substantially inextensible, or more concretely, the fabric is stretched by less than 20% in left and right direction when experiencing a stretching force of 45 cN or more in the same directions. Due to the hardly- or non-stretchy fabric, this region 128 can generate a higher reaction force against an external tensile force than other regions of the rear body 120 can. This region 128 is hereinafter referred to as “restricting portion.” The restricting portion 128 is located above a horizontal line LVL connecting the upper end LWP of the left side seam 113 with the upper end RWP of the right side seam 114. On the left side of the restricting portion 128, the rear surface 132 of the left sleeve 130 is sewn, and on the right side of the restricting portion 128, the rear surface 142 of the right sleeve 140 is sewn. When pulled by the wearer's shoulders, the restricting portion 128 generates tensile forces as resistance against the pull and thus prevents the wearer's shoulder blades from moving apart from each other. As a result, the wearer's shoulder blades, upper portions of upper arms, and shoulders are pulled backward and toward the center of his/her back. Therefore, it is difficult for the wearer to adopt the posture in which the shoulders extend forward to put the arms in front of the trunk.

The restricting portion is not limited to the region 128 between the rear surface 132 of the left sleeve 130 and the rear surface 142 of the right sleeve 140, but it may consist of a larger region or the entirety of the rear body 120.

Since the left sleeve 130 and the right sleeve 140 have shapes that are mirror images of each other, the following will explain the structure of the left sleeve 130 only. The structure of the right sleeve 140 could be easily understood by persons skilled in the art from the structure of the left sleeve 130.

The left sleeve 130 has a structure consisting of two pieces of fabric that are sewn on each other along an underarm line 133 and a dart 134 to form a cylinder. The dart 134 is a region where the pieces of fabric are tucked. The dart 134 extends across the rear surface 132 of the left sleeve 130 throughout the whole length thereof. As shown in FIG. 3, the direction of the dart 134 forms an angle α with the center plane CNP that bisects the rear body 120 in left and right direction. This angle α ranges preferably from about 70 degrees to about 110 degrees, or more preferably from about 80 degrees to about 100 degrees. Due to the presence of the dart 134, the rear surface 132 of the left sleeve 130 is rounded to fit close to the wearer's left arm. This prevents the left sleeve 130 from naturally riding up along the wearer's left arm toward his/her left shoulder.

The left sleeve 130 includes a cuff 135 and a left connecting portion 136. The cuff 135 corresponds to an open distal end of the cylindrical shape of the left sleeve 130, and the left connecting portion 136 corresponds to an open proximal end thereof. The left connecting portion 136 is a portion of the left sleeve 130 that is also called as sleeve cap and covers the wearer's left shoulder, and whose entirety is sewn on the left armhole 118.

At least the rear surface of the left connecting portion 136 (cf. sparsely dotted regions shown in FIG. 3) is a hardly- or non-stretchy region. The “hardly-stretchy region” is a region whose fabric is hardly stretched, or more concretely, a region stretched by 20% in the longitudinal direction of the left sleeve 130 when experiencing a stretching force in the same direction ranging from 45 cN to 600 cN, inclusive, or preferably ranging from 60 cN to 500 cN, inclusive, or more preferably, ranging from 80 cN to 400 cN, inclusive. The “non-stretchy region” is a region whose fabric is almost or substantially inextensible, or more concretely, a region stretched in the longitudinal direction of the left sleeve 130 by less than 20%, or more preferably less than 10%, when experiencing a stretching force of 45 cN in the same direction. The hardly- or non-stretchy region of the left sleeve 130 may range from the rear surface of the left connecting portion 136 to the front surface thereof. According to these structures, the left connecting portion 136 generates a higher reaction force against an external tensile force, thus hardly or never reducing the tensile force of the restricting portion 128. As a result, the garment upper body 100 can effectively prevent the forward motion of the wearer's shoulder blades, upper arms, and shoulders.

As shown in FIGS. 2 and 3, the lowest point LWP on a seam between the rear surface 132 of the left sleeve 130 and the rear body 120, which is hereinafter referred to as “first bottom point,” is positioned at a depth LUP below the lowest point LSP on a seam between the front surface 131 of the left sleeve 130 and the front body 110, which is hereinafter referred to as “first seam point.” The first seam point LSP is a portion of the seam located below the armpit. This depth LUP ranges preferably from 1 cm to 20 cm, inclusive. The presence of the depth LUP enables the fabric of the left sleeve 130 to have an enlarged region on a lower side of the rear surface 132, which allows the wearer to rotate the left arm around the left shoulder and raise it forward from the left side of the trunk. Thus, it is relatively easy for the wearer, without seriously harming the effect of the tensile force by the restricting portion 128, to rotate the left arm around the left shoulder and raise it forward from the left side of the trunk.

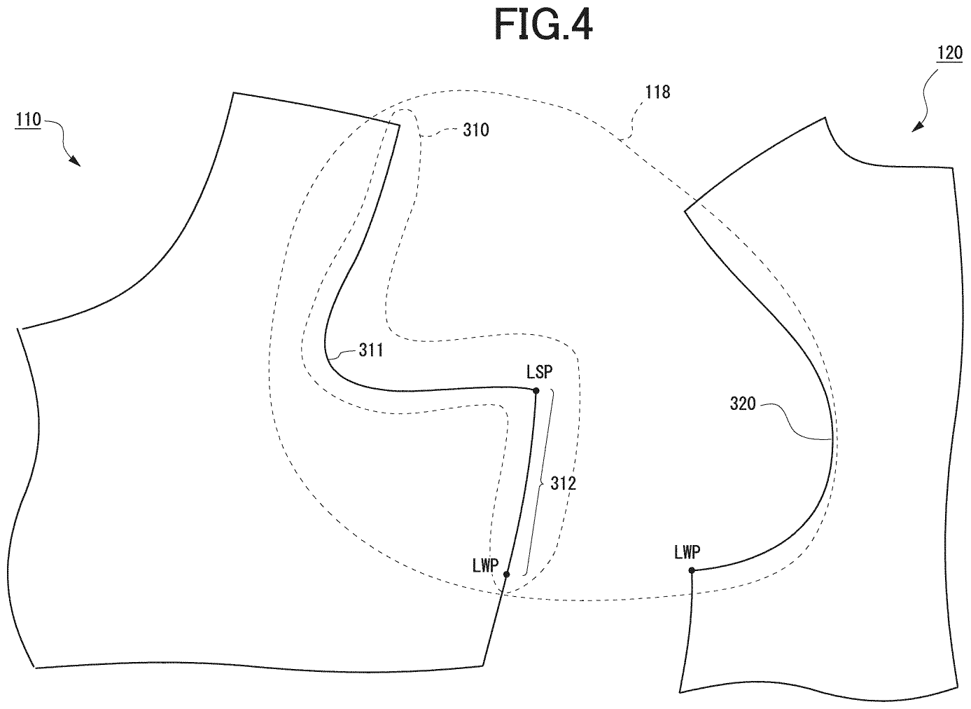

FIG. 4 is a partial development view of the left armhole 118 and its vicinity in the front body 110 and the rear body 120. Since a partial development view of the right armhole 119 and its vicinity is equivalent to a mirror image of FIG. 4, the following will explain the left armhole 118, and it is obvious for persons skilled in the art that the following explanation is also applicable to the right armhole 119.

When the front body 110 and the rear body 120 are separated along the seams, the left armhole 118 is divided into a front segment 310 and a rear segment 320. The front segment 310 is located at the upper left side of the front body 110 (which is shown in FIG. 4 at the upper right side thereof) and includes a left deeply-cut portion 311 and an upper-left side portion 312. The left deeply-cut portion 311 is shaped like a curve remained after the upper left corner of the front body 110 is deeply cut off, and is to be sewn on the front surface 131 of the left sleeve 130. The upper-left side portion 312 is a portion constituting an upper end of the left side seam 113 of the body and to be sewn on the rear surface 132 of the left sleeve 130. The rear segment 320 of the left armhole 118 is located at the upper left side of the rear body 120, shaped like a curve remained after the upper left corner of the rear body 120 is deeply cut off, and to be sewn on the rear surface 132 of the left sleeve 130.

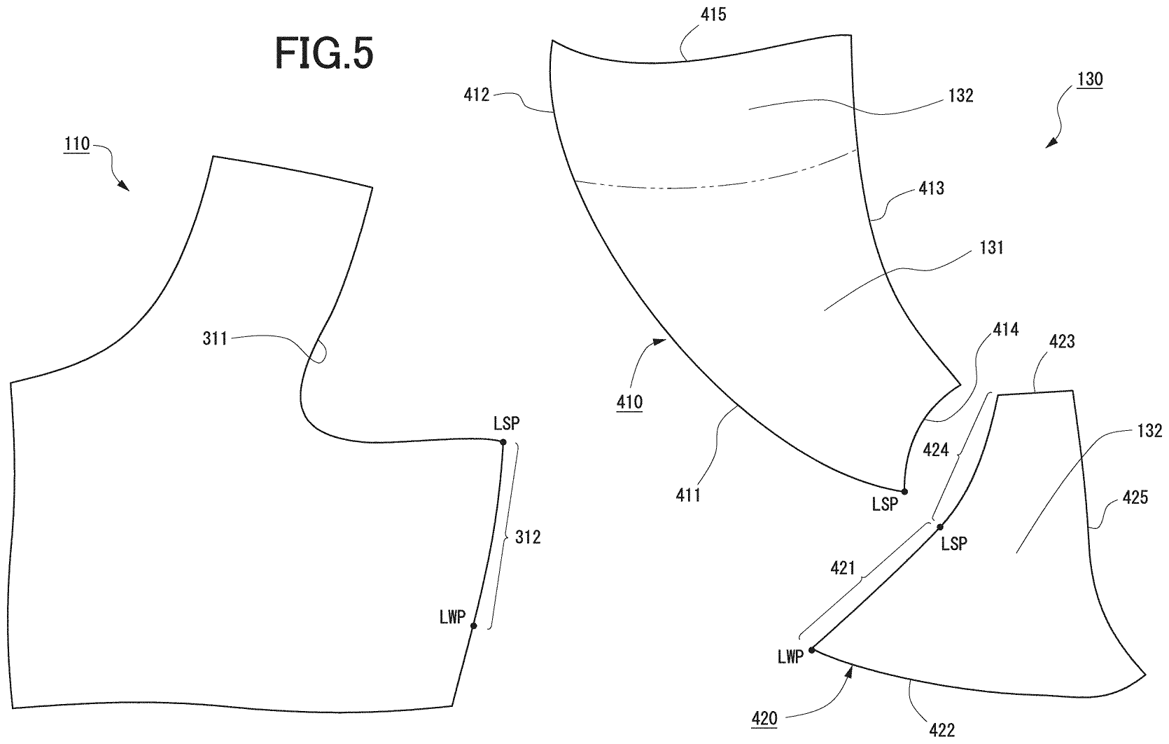

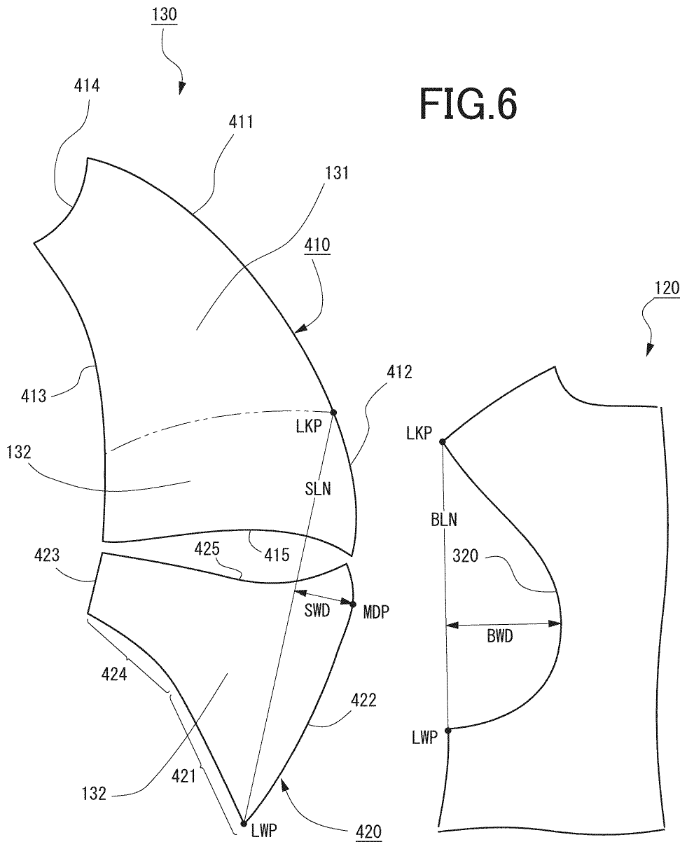

FIG. 5 is a partial development view of the left armhole 118 and its vicinity in the front body 110 and the left sleeve 130. FIG. 6 is a partial development view of the left armhole 118 and its vicinity in the rear body 120 and the left sleeve 130. Since partial development views of the right armhole 119 and its vicinity are equivalent to mirror images of FIGS. 5 and 6, the following will explain the left armhole 118 and the left sleeve 130, and it is obvious for persons skilled in the art that the following explanation is also applicable to the right armhole 119 and the right sleeve 140.

The left sleeve 130 has a structure consisting of an overarm portion 410 and an underarm portion 420 sewn on each other in a cylindrical shape. The overarm portion 410 forms the entirety of the front surface 131 of the left sleeve 130 and an upper region of the rear surface 132 thereof. The underarm portion 420 forms a lower region of the rear surface 132 of the left sleeve 130. One of seams between the overarm portion 410 and the underarm portion 420 extends as an underarm seam 133 of the left sleeve 130, and the other forms a dart 134 (cf. FIG. 3).

The circumference of the overarm portion 410 includes a front sleeve cap line 411, a rear sleeve cap line 412, a cuff line 413, an underarm line 414, and a seam line 415. The circumference of the underarm portion 420 includes a front sleeve cap line 421, a rear sleeve cap line 422, a cuff line 423, an underarm line 424, and a seam line 425. The front sleeve cap line 411 of the overarm portion 410 is sewn on the left deeply-cut portion 311 of the front body 110, and the front sleeve cap line 421 of the underarm portion 420 is sewn on the upper-left side portion 312 of the front body 110. The rear sleeve cap line 412 of the overarm portion 410 and the rear sleeve cap line 422 of the underarm portion 420 are sewn on the left armhole's rear segment 320 of the rear body 120. The underarm line 414 of the overarm portion 410 and the underarm line 424 of the underarm portion 420 are sewn on each other to form the underarm seam 133 of the left sleeve 130 (cf. FIGS. 2 and 3). The seam line 415 of the overarm portion 410 and the seam line 425 of the underarm portion 420 are sewn on each other to form the dart 134 of the left sleeve 130. As a result, the cuff line 413 of the overarm portion 410 and the cuff line 423 of the underarm portion 420 form the cuff 135 of the left sleeve 130.

A section MDP of the left armhole's rear segment 320 (cf. FIG. 6) farthest from a straight line BLN connecting both ends LKP and LWP of the left armhole's rear segment 320 is referred to as “deepest section”. The deepest section MDP of the left armhole's rear segment 320 is located to the right of the leftmost portion of the left shoulder seam 115 of the rear body 120, i.e. the right of the upper end LKP of the left armhole's rear segment 320. Compared to such a large curvature of the left armhole's rear segment 320, the rear sleeve cap lines 412 and 422 of the left sleeve 130 have smaller curvatures. That is, before the left sleeve 130 is sewn on the rear body 120, a width SWD of the rear sleeve cap lines 412 and 422 is smaller than a width BWD of the left armhole's rear segment 320: SWD<BWD; the width SWD of the rear sleeve cap lines 412 and 422 is the maximum distance of the rear sleeve cap lines 412 and 422 from a straight line SLN connecting both ends LKP and LWP of the entirety of the rear sleeve cap lines 412 and 422; the width BWD of the left armhole's rear segment 320 is the distance of the deepest section MDP from the straight line BLN connecting both the ends LKP and LWP of the left armhole's rear segment 320. Due to such a relationship of these widths SWD and BWD, the rear sleeve cap lines 412 and 422 cannot reach the left armhole's rear segment 320 until stretched to the right. As a result, the left sleeve 130, once sewed on the rear body 120, is naturally pitched more backward than the rear body 120.

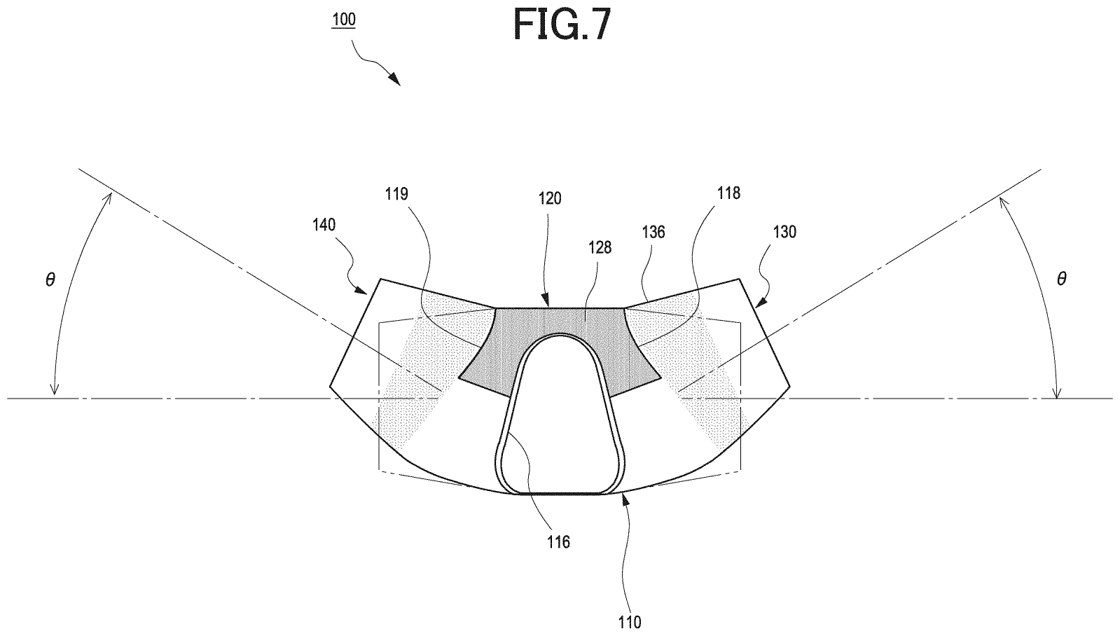

FIG. 7 is a plan view of a solid shape of the garment upper body 100. The “solid shape” of a garment means a three-dimensional shape of the garment when inflated with no portions stretched or folded. Solid shapes are generally designed to wrap and fit standard shapes of human body. As shown in FIG. 7, in the solid shape of the garment upper body 100, the direction of the left connecting portion 136 is tilted more backward than the direction of a standard left connecting portion. Thus, the solid shape of the entirety of the left sleeve 130 is tilted backward (diagonally backward left) at an angle θ relative to the solid shape of a left sleeve wrapping the left arm of a standard human body (cf. dashed-two dotted lines shown in FIG. 7). The same is true for the right sleeve 140. Since tilted backward in such a manner, both the sleeves 130 and 140 pull shoulder blades, upper portions of upper arms, and shoulders of a wearer backward and toward the center of his/her back. Accordingly, it is difficult for the wearer to adopt the posture in which the shoulders extend forward to put the arms in front of the trunk.

### Embodiment 2

In the garment upper body 100 according to embodiment 1 of the invention, the restricting portion 128 of the rear body 120 is woven with other regions thereof into a single fabric. However, a restricting portion according to the invention is not limited to such structure but may consist of an additional fabric attached to the rear body 120.

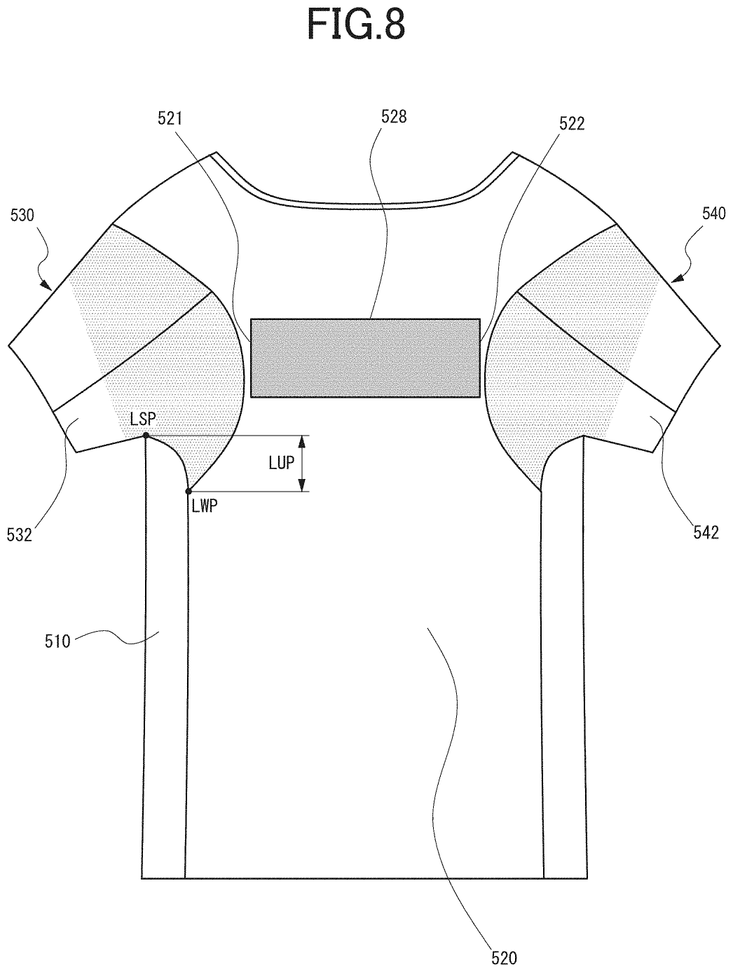

FIG. 8 is a rear elevation view of an upper body of a garment 500 according to embodiment 2 of the invention. This garment upper body 500 is a short-sleeved shirt, like that according to embodiment 1. The garment upper body 500 may be designed as a shirt with long or three-quarter sleeves. In the garment upper body 500, the entirety of a rear body 520 is comparable in elasticity to a front body 510, while a restricting portion 528 consists of an additional fabric attached to the rear body 520, which is hardly- or non-stretchy. The fabric of the restricting portion 528 has an elongated rectangular shape extending in left and right direction, whose left end 521 is sewn on a region of the rear body 520 inside the boundary between the rear surface 532 of the left sleeve 530 and the rear body 520, and whose right end 522 is sewn on a region of the rear body 520 inside the boundary between the rear surface 542 of the right sleeve 540 and the rear body 520. Since the restricting portion 528 is attached to the rear body 520, the wearer's shoulder blades are prevented from moving apart from each other, i.e. it is difficult for the wearer to adopt the posture in which the shoulders extend forward to put the arms in front of the trunk.

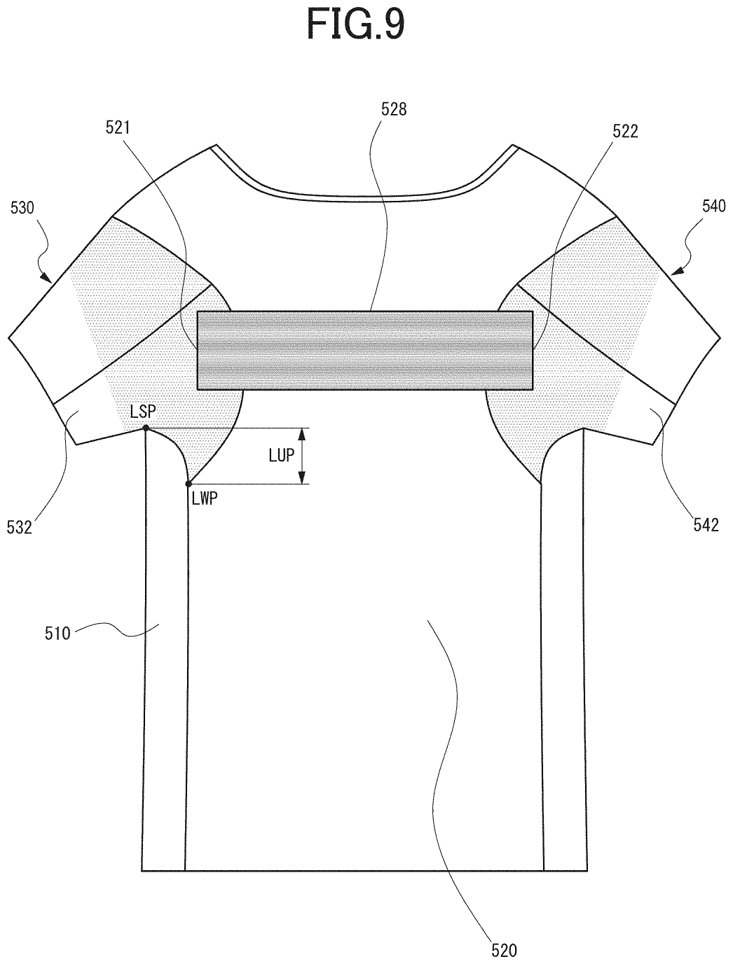

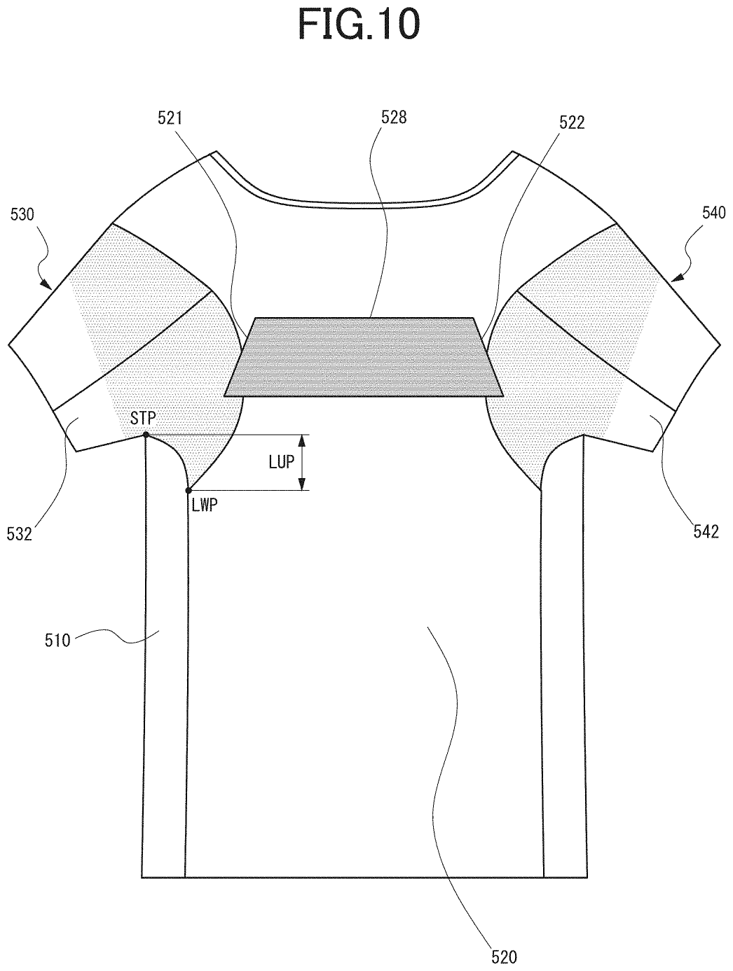

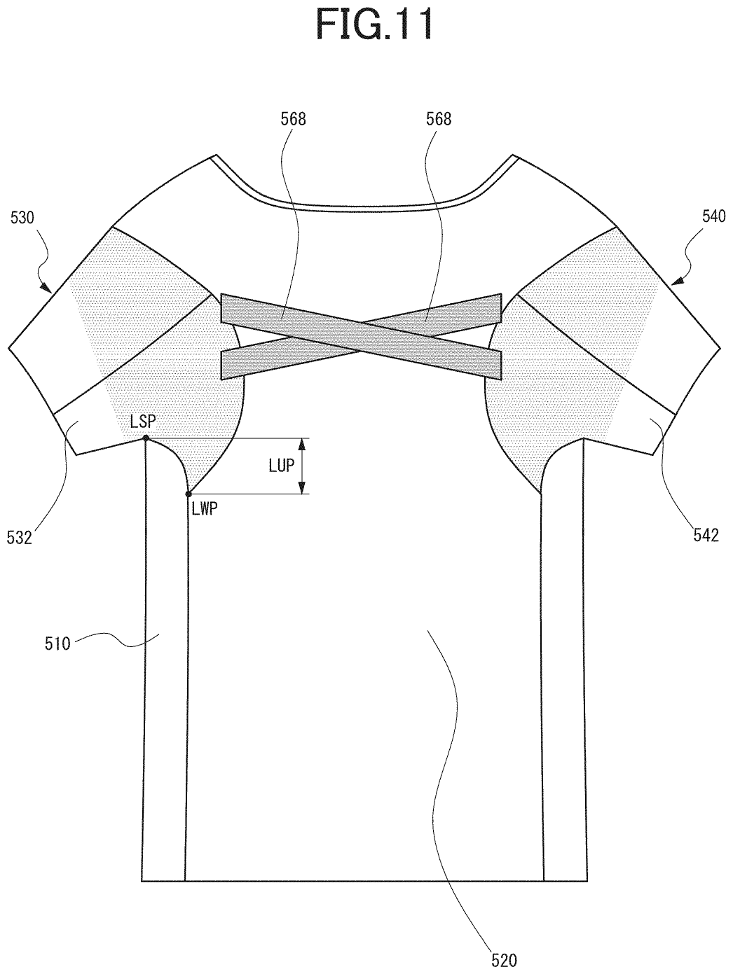

As shown in FIG. 9, the left end 521 of the restricting portion 528 may be sewn on the rear surface 532 of the left sleeve 530, and the right end 522 of the restricting portion 528 may be sewn on the rear surface 542 of the right sleeve 540. As shown in FIG. 10, the left end line 521 of the restricting portion 528 may cross the left armhole and be sewn on both the rear surface 532 of the left sleeve 530 and the rear body 520, and the right end line 522 of the restricting portion 528 may cross the right armhole and be sewn on the rear surface 542 of the right sleeve 540 and the rear body 520. A restricting portion according to the invention is limited to an additional single fabric, but it may consist of two or more fabrics. As shown in FIG. 11, two intersecting bands of fabric 568 may be attached as a restricting portion according to the invention. These bands 568 may be arranged in parallel to each other.

In view of the explanation described above, the invention can obviously have many variations and modifications. Accordingly, it should be understood that, within the scope of the claims attached to the specification, the invention can have embodiments other than those described in the specification.

1. An upper body of a garment comprising:
a body including a front body and a rear body;
a left sleeve including:
a first seam point that is the lowest point on a seam between a front surface of the left sleeve and the front body; and
a first bottom point that is the lowest point on a seam between a rear surface of the left sleeve and the rear body, the first bottom point being positioned below the first seam point;
a right sleeve including:
a second seam point that is the lowest point on a seam between a front surface of the right sleeve and the front body; and
a second bottom point that is the lowest point on a seam between a rear surface of the right sleeve and the rear body, the second bottom point being positioned below the second seam point;
and
a restricting portion located between the rear surfaces of the left and right sleeves and preventing left and right shoulder blades of a wearer from moving apart from each other.

2. The upper body of the garment according to claim 1, wherein the restricting portion includes a region of the rear body from a boundary between the rear body and the left sleeve to a boundary between the rear body and the right sleeve.

3. The upper body of the garment according to claim 1, wherein the restricting portion includes a fabric attached to the rear body and different from a fabric of the rear body.

4. The upper body of the garment according to claim 1, wherein
the left sleeve includes:
a left connecting portion connected to a left side of the body; and
a hardly-stretchy region that, when being stretched by 20% in a longitudinal direction of the left sleeve, increases a stretching force to 45 cN or more in the same direction and that extends at least within the rear surface of the left connecting portion,
and
the right sleeve includes:
a right connecting portion connected to a right side of the body; and
a hardly-stretchy region that, when being stretched by 20% in a longitudinal direction of the right sleeve, increases a stretching force to 45 cN or more in the same direction and that extends at least within the rear surface of the right connecting portion.

5. The upper body of the garment according to claim 4, wherein:
the hardly-stretchy region of the left sleeve ranges from the rear surface of the left connecting portion to a front surface thereof; and
the hardly-stretchy region of the right sleeve ranges from the rear surface of the right connecting portion to a front surface thereof.

6. The upper body of the garment according to claim 1, wherein
the left sleeve includes:
a left connecting portion connected to a left side of the body; and
a non-stretchy region that is stretched by less than 20% in the longitudinal direction of the left sleeve when experiencing a stretching force of 45 cN or more in the same direction and that extends at least within the rear surface of the left connecting portion;
and
the right sleeve includes:
a right connecting portion connected to a right side of the body; and
a non-stretchy region that is stretched by less than 20% in the longitudinal direction of the right sleeve when experiencing a stretching force of 45 cN or more in the same direction and that extends at least within the rear surface of the right connecting portion.

7. The upper body of the garment according to claim 6, wherein
the non-stretchy region of the left sleeve ranges from the rear surface of the left connecting portion to a front surface thereof; and
the non-stretchy region of the right sleeve ranges from the rear surface of the right connecting portion to a front surface thereof.

8. The upper body of the garment according to claim 2, wherein
the left sleeve includes:
a left connecting portion connected to a left side of the body; and
a hardly-stretchy region that, when being stretched by 20% in a longitudinal direction of the left sleeve, increases a stretching force to 45 cN or more in the same direction and that extends at least within the rear surface of the left connecting portion,
and
the right sleeve includes:
a right connecting portion connected to a right side of the body; and
a hardly-stretchy region that, when being stretched by 20% in a longitudinal direction of the right sleeve, increases a stretching force to 45 cN or more in the same direction and that extends at least within the rear surface of the right connecting portion.

9. The upper body of the garment according to claim 8, wherein:
the hardly-stretchy region of the left sleeve ranges from the rear surface of the left connecting portion to a front surface thereof; and
the hardly-stretchy region of the right sleeve ranges from the rear surface of the right connecting portion to a front surface thereof.

10. The upper body of the garment according to claim 3, wherein
the left sleeve includes:
a left connecting portion connected to a left side of the body; and
a hardly-stretchy region that, when being stretched by 20% in a longitudinal direction of the left sleeve, increases a stretching force to 45 cN or more in the same direction and that extends at least within the rear surface of the left connecting portion,
and
the right sleeve includes:
a right connecting portion connected to a right side of the body; and
a hardly-stretchy region that, when being stretched by 20% in a longitudinal direction of the right sleeve, increases a stretching force to 45 cN or more in the same direction and that extends at least within the rear surface of the right connecting portion.

11. The upper body of the garment according to claim 10, wherein:
the hardly-stretchy region of the left sleeve ranges from the rear surface of the left connecting portion to a front surface thereof; and
the hardly-stretchy region of the right sleeve ranges from the rear surface of the right connecting portion to a front surface thereof.

12. The upper body of the garment according to claim 2, wherein
the left sleeve includes:
a left connecting portion connected to a left side of the body; and
a non-stretchy region that is stretched by less than 20% in the longitudinal direction of the left sleeve when experiencing a stretching force of 45 cN or more in the same direction and that extends at least within the rear surface of the left connecting portion;
and
the right sleeve includes:
a right connecting portion connected to a right side of the body; and
a non-stretchy region that is stretched by less than 20% in the longitudinal direction of the right sleeve when experiencing a stretching force of 45 cN or more in the same direction and that extends at least within the rear surface of the right connecting portion.

13. The upper body of the garment according to claim 12, wherein
the non-stretchy region of the left sleeve ranges from the rear surface of the left connecting portion to a front surface thereof; and
the non-stretchy region of the right sleeve ranges from the rear surface of the right connecting portion to a front surface thereof.

14. The upper body of the garment according to claim 3, wherein
the left sleeve includes:
a left connecting portion connected to a left side of the body; and
a non-stretchy region that is stretched by less than 20% in the longitudinal direction of the left sleeve when experiencing a stretching force of 45 cN or more in the same direction and that extends at least within the rear surface of the left connecting portion;
and
the right sleeve includes:
a right connecting portion connected to a right side of the body; and
a non-stretchy region that is stretched by less than 20% in the longitudinal direction of the right sleeve when experiencing a stretching force of 45 cN or more in the same direction and that extends at least within the rear surface of the right connecting portion.

15. The upper body of the garment according to claim 14, wherein
the non-stretchy region of the left sleeve ranges from the rear surface of the left connecting portion to a front surface thereof; and
the non-stretchy region of the right sleeve ranges from the rear surface of the right connecting portion to a front surface thereof.
